(19) United States
(12) Plant Patent Application Publication
(10) Pub. No.: US 2015/0223379 P1
(43) Pub. Date: Aug. 6, 2015

(54) Avocado tree named 'LALI'

(50) ;

(71) Applicant: ACOSTA FARMS, INC., Miami, FL (US)

(72) Inventor: Alcides Acosta, Coral Gables, FL (US)

(73) Assignee: ACOSTA FARMS, INC., Miami, FL (US)

(21) Appl. No.: 13/999,231

(22) Filed: Jan. 31, 2014

(51) Int. Cl. A01H 5/00

(52) U.S. Cl. PLT/200

(57) ABSTRACT

The ‘Lali’ avocado is of medium size, average 14-18 ounces and being about 3.5 inches in diameter and 5 inches in length. The fruit matures and can be picked in late February and March. The fruit is an ovate berry having a seed that is tight in the cavity and is 2 to 2.5 inches long and dark brown in color.

### LATIN NAME OF THE GENUS AND SPECIES OF THE PLANT AND VARIETY DENOMINATION

Scientific Name: Persea Americana Mille var. ‘Lali’.

### BRIEF SUMMARY OF THE INVENTION

The tree is in a cultivated 85 acre avocado grove located at in Miami-Dade County, Florida, at 355014 Southwest 209^{th} Avenue, Miami, Fla. 33034. The tree is estimated to be 20 years old and has been in production for at least 10 years. Its origin is unknown. Its fruit can be stored at temperatures above 50° Fahrenheit.

Parentage is unknown as the tree was discovered, and is of a wild nature. An analysis of the microsatellite pattern from this avocado against 15 known varieties (Lula, Simmonds, Monroe, Choquette, Semil-34, Semil-43, Melendez, Hall, Booth8, Hardee, April, Carla, Pollock, Bernecker and Donnie) revealed no matches.

Asexual propagation was attained in 2010 in Miami-Dade County, Fla. when the plant was approximately 17 years old. Asexual reproduction was achieved by grafting cuttings of the tree onto new avocado seedlings. The fruit is ripened and is sufficient for harvesting in late February to late March. Lali presents resistance to carpospores purpurea pathogens.

### BRIEF DESCRIPTION OF THE DRAWINGS

FIG. 1 shows the leaves and branches at a lower portion of the Lali avocado tree.

FIG. 2 shows a closer view of the leaves and branches in FIG. 1.

FIG. 3 shows the middle portion of the Lali avocado tree.

FIG. 4 shows a closer view of the leaves of FIG. 3.

FIG. 5 shows a detailed view of the leaves of the Lali avocado tree.

FIG. 6 shows the density of leaves and branches of the Lali avocado tree.

FIG. 7 shows a view of an entire Lali avocado tree.

FIG. 8 shows a view of the upper level of the Lali avocado tree.

FIG. 9 shows a view of the top level of the Lali avocado tree.

FIG. 10 shows the scale in inches of a typical fruit of the Lali avocado tree measured against a ruler.

FIG. 11 shows the color and internal configuration of the fruit and the seed of the Lali avocado tree.

### DETAILED BOTANICAL DESCRIPTION

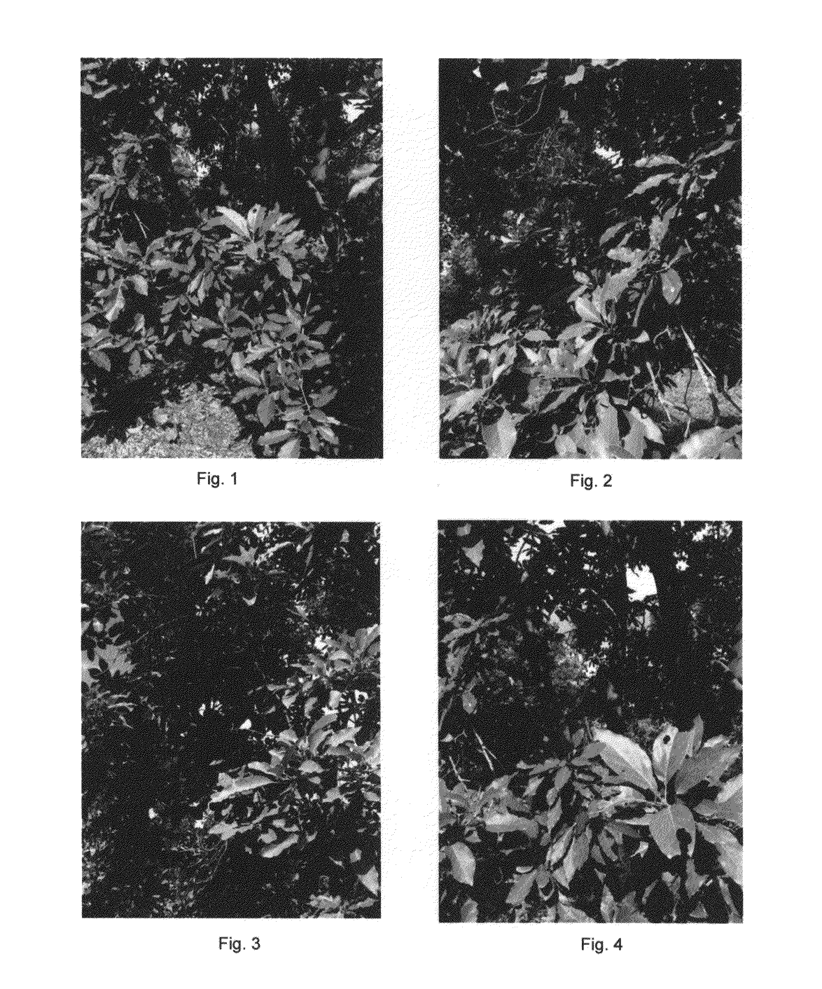

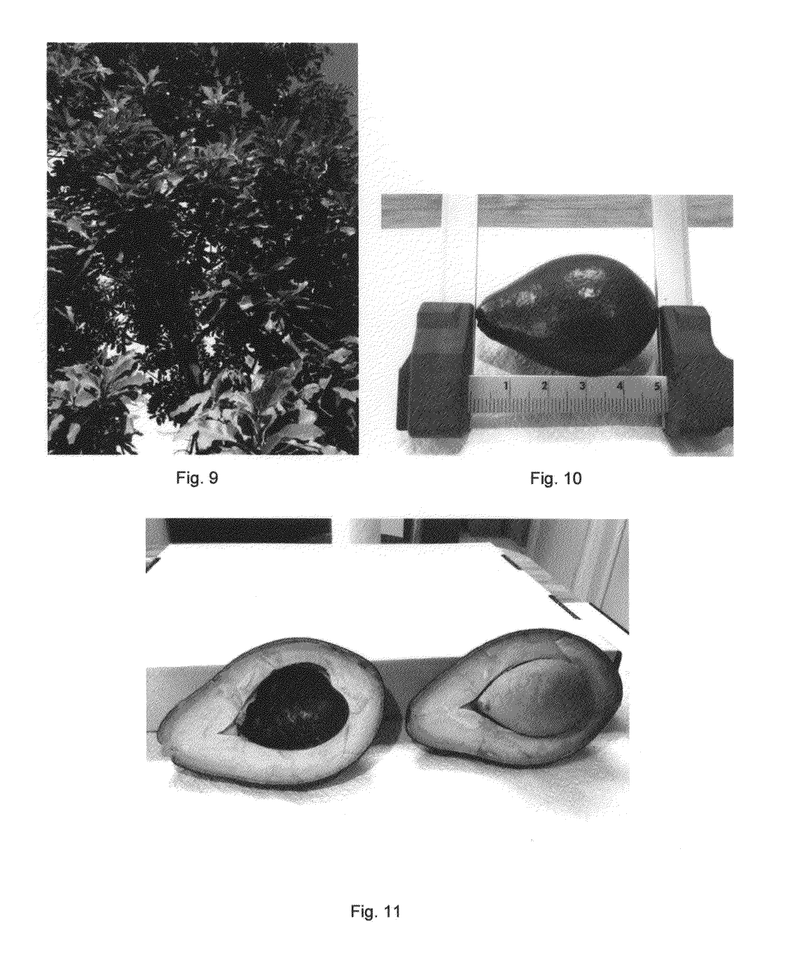

The tree presents a vigorous upright growth with a spread of 18′ and 30′ high, as shown in FIG. 7. As shown in FIGS. 1, 4 and 6, the diameter of the main trunk is 23″-25″. The main trunk splits into 4 sub trunks or branches, each about 3-4 inches in width with the vertical sub trunk splitting into smaller branches, FIG. 9. The bark of the trunk is light in color with new shoots being green in color and the foliage being dark green at maturity and burgundy at a young stage.

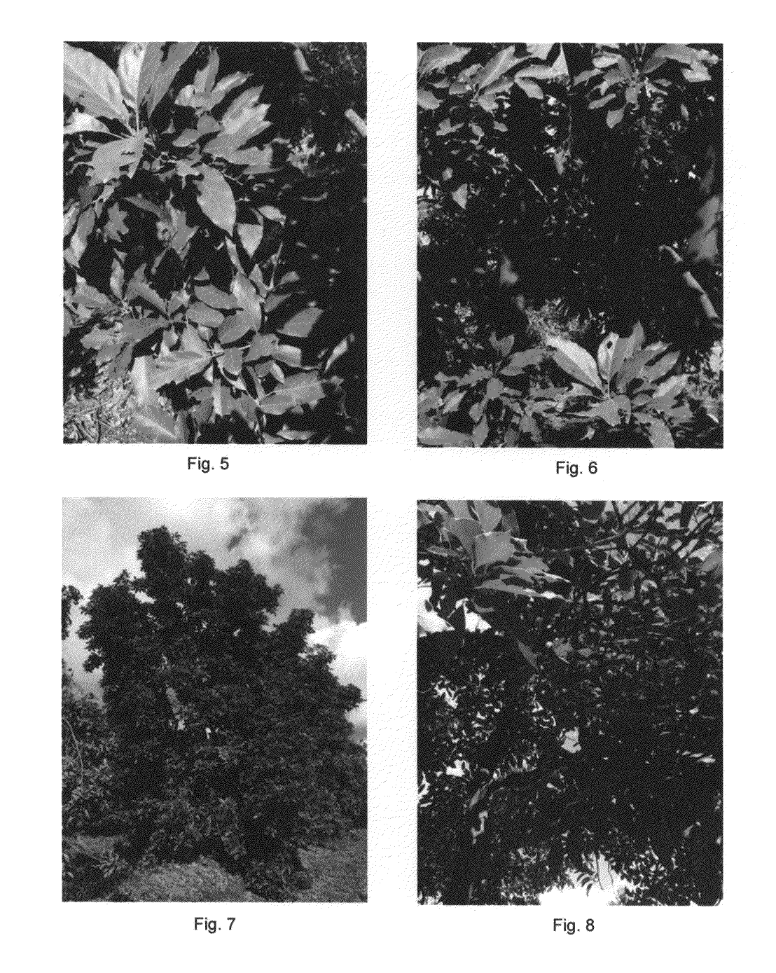

The leaves are light green when young, becoming dark green when mature, are 3.5″ wide by 9″ in length as shown in FIG. 5, and have a smooth surface that becomes leathery in texture as they mature, as appears in FIGS. 3, and 5. The foliage is sparse near the ground, FIGS. 1 and 2, becoming more dense toward the upper regions of the tree, FIGS. 7, 8 and 9. Typical observed petiole diameter is 3.5-3.7 mm. Buds are yellow-green in color, have an average length of 7-11 mm, average width of 3.75-4 mm, and a Lancelot to oblong-Lancelot shape with a rounded base and acute tip. Pedicels are green-yellow in color and average 6 mm in length and 1.3 mm in width with an average diameter of 1-2 mm. Peduncles range from 42 mm to 153 mm and average about 87 mm and are yellow green in color. Typical observed flower depth is 7.5-8 mm.

The flowers of this plant have 3 lobes that are approximately 6.5-6.7 mm in length and 2 mm in width. Margins are smooth and uniform, and the texture is smooth. Densely silk tomentose is found on both surfaces. The shape is apex acute with a flattened base.

As shown in FIG. 10, the fruit shape is an ovate berry that matures in late February, and that averages 3.5″ in diameter and 5″ in length. As shown in FIG. 11, the interior of the fruit is seen to have a mesocarp color that is green near the shell and yellow near the seed cavity. The seed is round, weighing about 6.9-7.2 oz, depending on overall fruit size, having a diameter between 2.0 and 2.5″. The seed is tight in the cavity, is 2.5″ to 3.5″ long, and is dark brown in color.

The plant produces fruit at a medium-high volume, averaging about 250-300 lbs per year. The tree's winter hardness is 9B. Avocado shelf life is several weeks, and the fruit can be stored above 50° F. The fruit naturally ripens once placed at room temperature or about 5-8 days after harvesting.

The flowers are not fragrant. The tree blooms in February and the flowers are of the B type. Fruit matures and can be picked in late February or March of the following year. The fruit weights are between 14 and 18 ounces with the diameter averaging 5 inches.

1. A new and distinct variety of avocado tree substantially as described and illustrated and characterized as to novelty by its overall good eating qualities, its medium size and its being ready to be picked in late February and March.
